(19) United States
(12) Patent Application Publication
(10) Pub. No.: US 2026/0069301 A1
(43) Pub. Date: Mar. 12, 2026

(54) CATHETER FOR USE WITH CLOT TREATMENT SYSTEMS

(71) Applicant: Inari Medical, Inc., Irvine, CA (US)

(72) Inventors: Aaron Dederich, San Clemente, CA (US); Jakub Marek Truty, Anaheim, CA (US); Brian Edward Merritt, San Clemente, CA (US); Thien Mai, Santa Ana, CA (US)

(21) Appl. No.: 19/387,957

(22) Filed: Nov. 13, 2025

Related U.S. Application Data
(63) Continuation of application No. 18/408,177, filed on Jan. 9, 2024, now Pat. No. 12,496,081.
(60) Provisional application No. 63/437,885, filed on Jan. 9, 2023.

(51) Int. Cl. A61B 17/221; A61B 17/00; A61B 17/22

(52) U.S. Cl. CPC A61B 17/221; A61B 2017/00336; A61B 2017/00367; A61B 2017/00982; A61B 2017/22038; A61B 2017/2215

(57) ABSTRACT

Disclosed herein are catheters for use with clot treatment systems, and associated devices and methods. The catheter can include an outer sheath, an outer sheath drive component operably coupled to the outer sheath, a guidewire sheath slidably disposed within the outer sheath, a guidewire sheath drive component operably coupled to the guidewire sheath and slidably disposed within the outer sheath drive component, and a clot treatment component. During a clot treatment procedure, the outer sheath drive component can be moved proximally and/or the guidewire sheath drive component can be moved distally to cause corresponding movement of the outer sheath and/or guidewire sheath, respectively, and transition the catheter between a first configuration, in which the clot treatment component can be positioned within the outer sheath, and a second configuration, in which at least part of the clot treatment component can extend beyond the outer sheath.

### CROSS-REFERENCE TO RELATED APPLICATIONS

This is a continuation of U.S. Patent Application No. 18/408,177, filed January 9, 2024, and titled “CATHETER FOR USE WITH CLOT TREATMENT SYSTEMS,” which claims priority to U.S. Provisional Patent Application No. 63/437,885, filed January 9, 2023, and titled “CATHETER FOR USE WITH CLOT TREATMENT SYSTEMS,” each of which is incorporated herein by reference in its entirety.

### TECHNICAL FIELD

The present technology generally relates to thrombectomy catheters for use with clot treatment systems, and associated devices and methods.

### BACKGROUND

Thromboembolic events are characterized by an occlusion of a blood vessel. Thromboembolic disorders, such as stroke, pulmonary embolism, heart attack, peripheral thrombosis, atherosclerosis, and the like, affect many people. These disorders are a major cause of morbidity and mortality.

When an artery is occluded by a clot, tissue ischemia develops. The ischemia will progress to tissue infarction if the occlusion persists. However, infarction does not develop or is greatly limited if the flow of blood is reestablished rapidly. Failure to reestablish blood flow can accordingly lead to the loss of limb, angina pectoris, myocardial infarction, stroke, or even death.

In the venous circulation, occlusive material can also cause serious harm. Blood clots can develop in the large veins of the legs and pelvis, a common condition known as deep venous thrombosis (DVT). DVT commonly occurs where there is a propensity for stagnated blood (e.g., long-distance air travel, immobility, etc.) and clotting (e.g., cancer; recent surgery, such as orthopedic surgery, etc.). DVT can obstruct drainage of venous blood from the legs, leading to swelling, ulcers, pain, and infection. DVT can also create a reservoir in which blood clots can collect and then travel to other parts of the body, including the heart, lungs, brain (which may cause a stroke), abdominal organs, and/or extremities.

Various thrombectomy devices exist for removing occlusive material (e.g., blood clots) to reestablish blood flow within a patient. Thrombectomy devices typically have a catheter containing a mechanical clot treatment component for engaging and removing occlusive material lodged in a blood vessel. Such thrombectomy devices are often delivered intravascularly to the occlusive material over a guidewire, which often extends through the entire length of the catheter’s lumen. The guidewire can be too short for some procedures such that there is insufficient length on the guidewire to deliver the thrombectomy device to the occlusive material. Additionally, long thrombectomy devices can require excessive time for exchanges over the guidewire. This can complicate the thrombectomy procedure, such as inadequately removing the occlusive material or otherwise failing to reestablish sufficient blood flow within the patient.

### BRIEF DESCRIPTION OF THE DRAWINGS

Many aspects of the present technology can be better understood with reference to the following drawings. The accompanying Figures depict embodiments of the present technology and are not intended to be limiting of its scope unless expressly indicated. The sizes of various depicted elements are not necessarily drawn to scale, and these various elements may be enlarged to improve legibility. Component details may be abstracted in the Figures to exclude details such as the position of components and certain precise connections between such components when such details are unnecessary for a complete understanding of how to make and use the present technology. Furthermore, components can be shown as transparent in certain views for clarity of illustration only and not to indicate that the component is necessarily transparent. Many of the details, dimensions, angles, and other features shown in the Figures are merely illustrative of particular embodiments of the disclosure. Accordingly, other embodiments can have other details, dimensions, angles, and features without departing from the present technology. In addition, those of ordinary skill in the art will appreciate that further embodiments of the present technology can be practiced without several of the details described below.

FIGS. 1A and 1B are side views of a clot treatment catheter in a first configuration and a second configuration, respectively, in accordance with embodiments of the present technology.

FIG. 1C is a partially-schematic cross-sectional view of the catheter of FIGS. 1A and 1B taken along line 1C–1C of FIG. 1A in accordance with embodiments of the present technology.

FIGS. 2A and 2B are enlarged side views of a portion of the clot treatment catheter of FIGS. 1A and 1B in the first and second configurations, respectively, in accordance with embodiments of the present technology.

FIGS. 3A and 3B are side views of another clot treatment catheter in a first configuration and a second configuration, respectively, in accordance with embodiments of the present technology.

FIG. 3C is a partially-schematic cross-sectional view of the clot treatment catheter of FIGS. 3A and 3B taken along line 3C–3C of FIG. 3A in accordance with embodiments of the present technology.

FIGS. 4A and 4B are side views of the clot treatment catheter of FIGS. 1A-1C in a first configuration and a second configuration, respectively, in accordance with embodiments of the present technology.

FIG. 5 is a side view of the clot treatment catheter of FIGS. 3A and 3B in the second configuration in accordance with embodiments of the present technology.

FIGS. 6A and 6B are side cross-sectional views of the clot treatment catheter of FIGS. 1A and 1B, respectively, in accordance with embodiments of the present technology.

FIG. 7 is a side view of the clot treatment catheter of FIGS. 3A and 3B in the second configuration in accordance with embodiments of the present technology.

FIG. 8 side view of the clot treatment catheter of FIGS. 3A and 3B in the second configuration in accordance with embodiments of the present technology.

### DETAILED DESCRIPTION

The present technology is generally directed to thrombectomy catheters for use with clot treatment systems, and associated devices and methods. In some of the embodiments described in detail below, a thrombectomy catheter (“catheter”) can include an outer sheath, an outer sheath drive component operably coupled to the outer sheath, a guidewire sheath slidably disposed within the outer sheath, and a guidewire sheath drive component operably coupled to the guidewire sheath and slidably disposed within the outer sheath drive component. Additionally, the catheter can include a clot treatment component. The clot treatment component can be coupled to the guidewire sheath and positionable within the outer sheath. In these and other embodiments, the catheter can include at least one clot treatment drive component operably coupled to the clot treatment component. The clot treatment drive component can be positioned between (e.g., radially between, telescopically between, and the like) the outer sheath drive component and the guidewire sheath drive component.

During a clot treatment procedure (e.g., removing occlusive material), the catheter can be transitioned between a first (e.g., sheathed) configuration in which the clot treatment component is contained within the outer sheath and a second (e.g., deployed) configuration in which the clot treatment component is deployed from within the outer sheath by moving the outer sheath and/or the guidewire sheath relative to each other. For example, to transition the catheter from the first configuration to the second configuration, a user can move the guidewire sheath drive component in a first direction (e.g., distally, a distal direction) relative to the outer sheath to cause a corresponding movement of the guidewire sheath and thereby extend at least a portion of the guidewire sheath outwardly from within the outer sheath. The clot treatment component can be coupled to or positioned around the portion of the guidewire sheath such that extending this portion of the guidewire sheath outwardly from within the outer sheath can allow the clot treatment component to expand or be deployed. Additionally, or alternatively, to transition the catheter from the first configuration to the second configuration, the user can move the outer sheath drive component in a second direction (e.g., proximally) relative to the guidewire sheath to cause a corresponding movement of the outer sheath and thereby withdraw the outer sheath over the guidewire sheath, e.g., to expose the portion of the guidewire sheath. To transition the catheter from the second configuration to the first configuration, the movement of the guidewire sheath drive component and/or the outer sheath drive component can be reversed, e.g., the user can move the guidewire sheath drive component in the second direction and/or the outer sheath drive component in the first direction.

The clot treatment component can be fully sheathed within the outer sheath in the first configuration, e.g., for delivery through a blood vessel to the site of an occlusion. In the second configuration, at least part of the clot treatment component can be deployed from or extend beyond the outer sheath. For example, in the second configuration the clot treatment component can be positioned for engaging an occlusion during a clot treatment. In some embodiments, the user can deploy, sheath, and/or change at least one dimension (e.g., length, width, diameter, cross-sectional area, and the like) and/or change at least one characteristic or mechanical property (e.g., radial force, tension, compression, shape, and the like) of the clot treatment component by moving the clot treatment drive component.

In some aspects of the present technology, the catheters can occupy a relatively short length of a guidewire, such that an increased length of the guidewire is available, for example, to allow additional clot treatment devices to be intravascularly delivered within the patient. This is expected to reduce at least some complications that may arise during clot treatment and/or removal procedures, such as when there is an insufficient length of the guidewire available for the user to adequately address the patient’s clot. Additionally, or alternatively, at least some of the catheters are expected to be easier for a user to transition between configurations, such that the user can more readily/easily perform multiple passes at clot material during a procedure. For example, at least some of the catheters can be configured such that the clot treatment component can be completely or at least partially re-sheathed after the clot treatment component has been deployed. In these and other embodiments, the catheters of the present technology are expected to occupy and/or engage a shorter length of a guidewire compared to other clot treatment devices, which can enable the catheter to be more readily/easily loaded and unloaded from the guidewire when performing multiple passes at clot material within the patient.

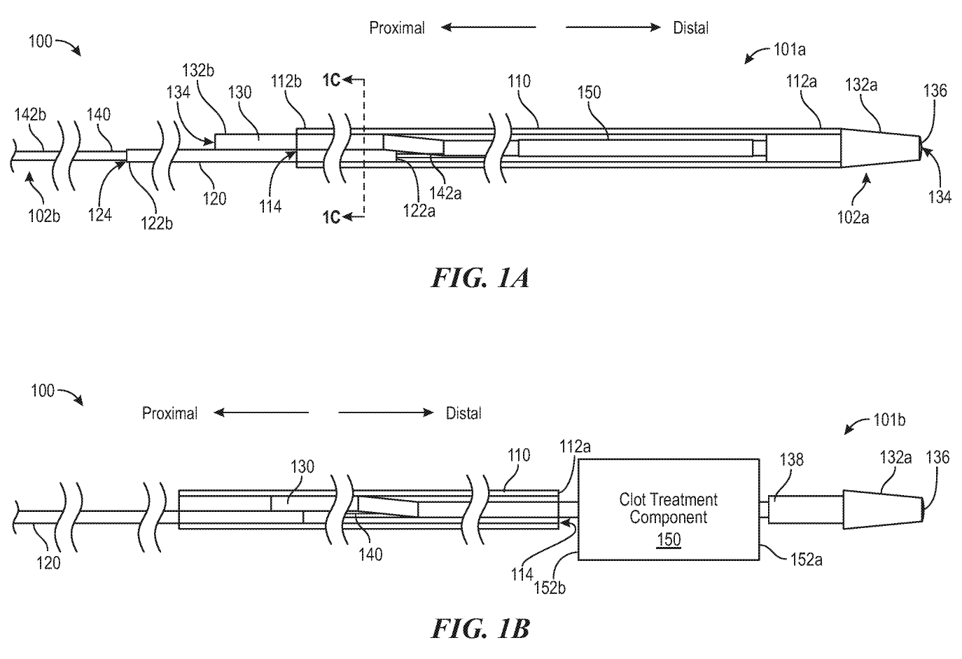

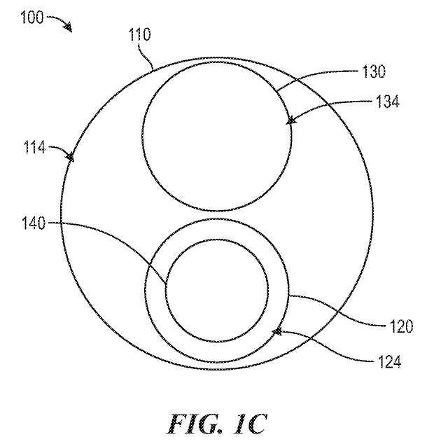

Certain details are set forth in the following description and in FIGS. 1A–8 to provide a thorough understanding of various embodiments of the present technology. In other instances, well-known structures, materials, operations, and/or systems often associated with intravascular procedures, blood filtering, clot removal procedures, catheters, and the like are not shown or described in detail in the following disclosure to avoid unnecessarily obscuring the description of the various embodiments of the technology. Those of ordinary skill in the art will recognize, however, that the present technology can be practiced without one or more of the details set forth herein and/or with other structures, methods, components, and so forth. Moreover, although many of the devices and systems are described herein in the context of removing and/or treating clot material from a vessel, the present technology can be used to remove and/or treat any unwanted material within a vessel in addition or alternatively to clot material, such as thrombi, emboli, plaque, etc. Accordingly, the terms “clot” and “clot material” as used herein can refer to any of the foregoing materials within a vessel.

The terminology used below is to be interpreted in its broadest reasonable manner, even though it is being used in conjunction with a detailed description of certain examples of embodiments of the technology. Indeed, certain terms may even be emphasized below; however, any terminology intended to be interpreted in any restricted manner will be overtly and specifically defined as such in this Detailed Description section.

With regard to the terms “distal” and “proximal” within this description, unless otherwise specified, the terms can reference a relative position of the portions of a catheter subsystem with reference to an operator and/or a location in the vasculature. Also, as used herein, the designations “rearward,” “forward,” “upward,” “downward,” and the like are not meant to limit the referenced component to a specific orientation. It will be appreciated that such designations refer to the orientation of the referenced component as illustrated in the Figures; the systems of the present technology can be used in any orientation suitable to the user.

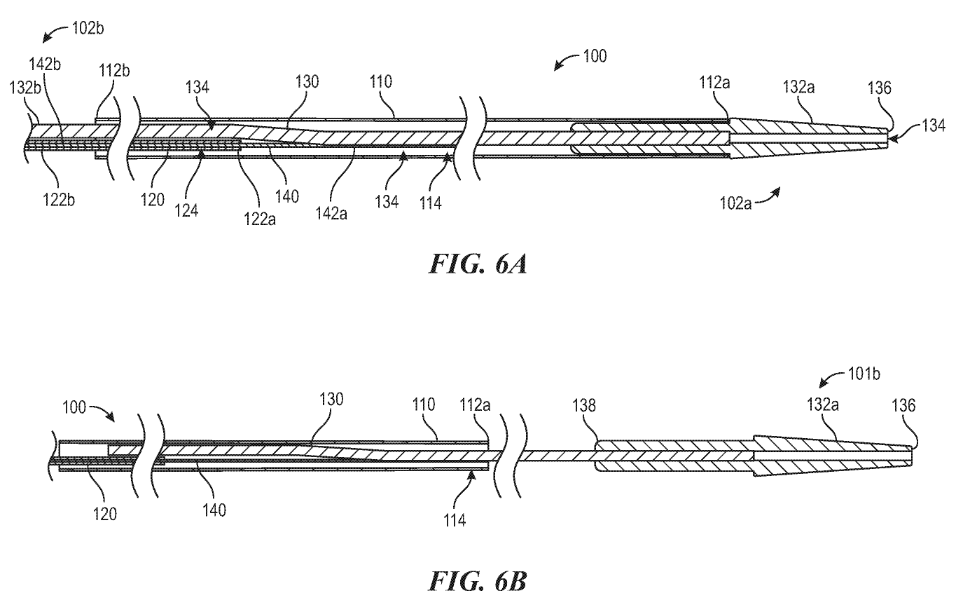

FIGS. 1A and 1B are side views of a clot treatment catheter (“catheter 100”) in a first configuration 101a and a second configuration 101b, respectively, in accordance with embodiments of the present technology. FIG. 1C is a partially-schematic cross-sectional view of the catheter 100 taken along line 1C–1C of FIG. 1A in accordance with embodiments of the present technology. FIGS. 6A and 6B are cross-sectional views of the catheter 100 in the first configuration 101a and the second configuration 101b, respectively, in accordance with embodiments of the present technology. In FIGS. 1A, 1B, 6A, and 6B, aspects of the catheter 100 are illustrated as transparent for clarity.

In some embodiments, the catheter 100 includes a first or distal end portion 102a and a second or proximal end portion 102b (the various ends/end portions described herein are not visible in the plane of FIG. 1C). During a clot treatment procedure, the catheter 100 can be advanced intravascularly to a treatment site within a blood vessel of a patient P. In at least some embodiments, for example, the catheter 100 is advanced though the vasculature until all or part of the clot treatment component 150 (not shown in FIGS. 6A and 6B) is positioned in or distal to clot material within the blood vessel.

The catheter 100 can include one or more sheaths and/or components. In some embodiments, the catheter 100 includes an outer sheath 110, an outer sheath drive component 120, a guidewire sheath 130, a guidewire sheath drive component 140, and a clot treatment component 150 (shown schematically). Referring to FIGS. 1A and 6A, the outer sheath 110 can include an elongated tube or member comprising a first or distal end portion 112a, a second or proximal end portion 112b opposite the distal end portion 112a, and a lumen 114 (“the outer sheath lumen 114”) extending from the distal end portion 112a to the proximal end portion 112b. The outer sheath drive component 120 can include an elongated tube or member comprising a distal end portion 122a, a proximal end portion 122b opposite the distal end portion 122a, and a lumen 124 (“the outer sheath drive component lumen 124”) extending from the distal end portion 122a to the proximal end portion 122b. At least part of the outer sheath drive component 120 (e.g., at least part of the distal end portion 122a) can be positioned within the outer sheath lumen 114 and coupled to the outer sheath 110. The proximal end portion 122b of the outer sheath drive component 120 can extend proximally from the proximal end portion 112b of the outer sheath 110 and/or can be integrated with a handle or other user interface, for example, to allow a user to grasp, manipulate, move, etc. the outer sheath drive component 120 to thereby move the outer sheath 110 relative to one or more other portions of the catheter 100.

The guidewire sheath 130 can be positioned at least partially within the outer sheath lumen 114, for example, proximate/adjacent to the outer sheath drive component 120. The guidewire sheath 130 can comprise a tube having a first or distal end or tip portion 132a (“distal tip 132a”), a second or proximal end portion 132b, and a lumen 134 (“guidewire sheath lumen 134”) extending from the distal tip 132a to the proximal end portion 132b. The distal tip 132a can be integral with the guidewire sheath 130 or a separate component coupled to a distal end of the guidewire sheath 130. The distal tip 132a of the guidewire sheath 130 can define a distal terminus 136 of the catheter 100. The guidewire sheath lumen 134 can be configured to receive a guidewire (not shown) therethrough. In at least some embodiments, for example, the guidewire can be inserted within the guidewire sheath lumen 134 via the distal tip 132a and extend fully beyond the proximal end portion 132b of the guidewire sheath 130. The guidewire sheath 130 can be relatively short in length. For example, the guidewire sheath 130 can be shorter than an overall length of the catheter 100 between the distal and proximal end portions 102a, 102b. Additionally, or alternatively, the proximal end 122b of the outer sheath drive component 120 can extend proximally beyond the proximal end 132b of the guidewire sheath 130 in the first and second configurations 101a, 101b. In some aspects of the present technology, relatively short guidewire sheaths 130 are expected to allow an increased length of a guidewire to be available for intravascular delivery of additional clot treatment devices within a patient. This, in turn, can reduce intravascular crowding, improve intravascular navigability, and/or increase the amount of clot material that can be removed from the patient.

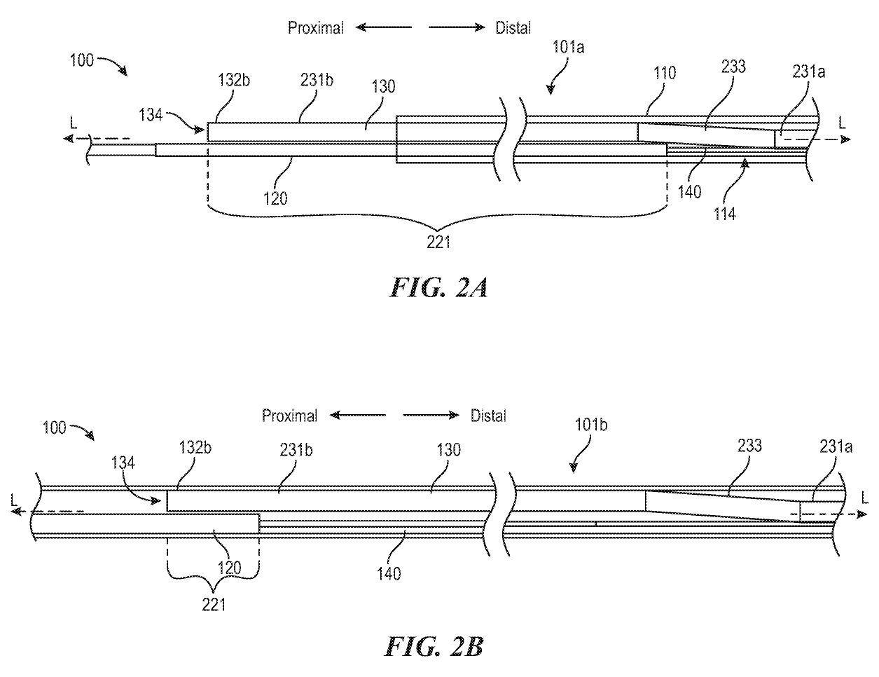

The guidewire sheath drive component 140 can be positioned at least partially within the outer sheath drive component lumen 124 and can be coupled to the guidewire sheath 130. The guidewire sheath drive component 140 can include an elongated shaft, tube, or member comprising a first or distal end portion 142a and a second or proximal end portion 142b opposite the distal end portion 142a. The distal end portion 142a of the guidewire sheath drive component 140 extends distally from the distal end portion 122a of the outer sheath drive component 120 and is coupled to the guidewire sheath 130, as shown and described in further detail with reference to FIGS. 2A and 2B. The proximal end portion 142b can extend proximally from the proximal end portion 122b of the outer sheath drive component 120 and/or can be integrated with a handle or user interface, for example, to allow a user to grasp, manipulate, move, etc. the guidewire sheath drive component 140 to thereby move the guidewire sheath 130 relative to one or more other portions of the catheter 100.

The clot treatment component 150 can comprise one or more of the clot treatment components described in detail in U.S. Pat. Nos. 8,784,434; 10,342,571; and/or 10,912,577; the entireties of which are hereby incorporated by reference. In at least some embodiments, for example, the clot treatment component 150 can include one or more stents, baskets, disks, spheres, funnels, balloons, and/or tubes, individual ones of which can be a laser cut, braided, extruded, woven component, etc. The clot treatment component 150 can be positioned within the outer sheath lumen 114, for example, between the interior surface of the outer sheath 110 and the exterior surface of the guidewire sheath 130. Referring to FIG. 1B, which shows the clot treatment component 150 in a deployed or expanded configuration, the clot treatment component 150 can include a distal end portion 152a and a proximal end portion 152b opposite the distal end portion 152a. The clot treatment component 150 can be coupled to the catheter 100, e.g., at one or both of the end portions 152a, 152b and/or at one or more other suitable portions of the clot treatment component 150 between the end portions 152a,b. In at least some embodiments, for example, the distal end portion 152a of the clot treatment component 150 can be coupled to the guidewire sheath 130 (e.g., the distal tip 132a), and/or any other suitable sheath and/or component of the catheter 100. Additionally, or alternatively, the proximal end portion 152b of the clot treatment component 150 can be coupled to the guidewire sheath 130, and/or any other suitable sheath and/or component of the catheter 100. In embodiments where one end (e.g., the distal end portion 152a) of the clot treatment component 150 is coupled to the guidewire sheath 130, and/or another suitable sheath and/or component of the catheter 100, the other end (e.g., the proximal end portion 152b) of the clot treatment component 150 can move or slide relative to the catheter 100, for example, along the exterior surface of the guidewire sheath 130 and/or at least partially between the distal end portion 112a of the outer sheath 110 and a proximal end portion 138 of the distal tip 132a. In at least some embodiments, the clot treatment component 150 is not coupled to the catheter 100 (e.g., free floating) and both the distal end portion 152a and the proximal end portion 152b can move or slide relative to each other and/or the catheter 100, e.g., while being retained on the guidewire sheath 130 via mechanical interference with the proximal end portion 138 of the distal tip 132a and/or the distal end portion 112a of the outer sheath 110. In these and other embodiments, the clot treatment component 150 can be configured to deploy/expand (e.g., radially deployed/expanded, longitudinally deployed/expanded, and the like) when the catheter 100 transitions from the first configuration 101a (FIG. 1A) to the second configuration 101b (FIG. 1B). The clot treatment component 150 can expand automatically, for example, in response to movement (e.g., by the user) of one or more of the sheaths and/or other components of the catheter 100.

Referring to FIGS. 1A, 1B, 6A, and 6B together, during a clot treatment procedure, the catheter 100 can be transitioned between the first configuration 101a (FIG. 1A) and the second configuration 101b (FIG. 1B) by moving one or more of the catheter’s components and/or sheaths. As shown in the embodiment illustrated in FIG. 1B, for example, the outer sheath drive component 120 has been moved proximally relative to the guidewire sheath 130 to move the outer sheath 110 in the proximal direction and/or away from the distal terminus 136. This transitions the catheter 100 from the first configuration 101a to and/or at least partially toward the second configuration 101b. As the outer sheath 110 moves proximally, the clot treatment component 150 is progressively exposed in a distal-to-proximal motion that allows the clot treatment component 150 to deploy/expand from the first (e.g., delivery) state (FIG. 1A) to and/or at least partially toward the second (e.g., deployed) state (FIG. 1B). In the embodiment illustrated in FIG. 1A, for example, the clot treatment component 150 is positioned completely within the outer sheath lumen 114, between the interior surface of the outer sheath 110 and the exterior surface of the guidewire sheath 130, such that the outer sheath 110 restricts expansion (e.g., radial expansion, longitudinal expansion, etc.) of the clot treatment component 150. Accordingly, moving the outer sheath 110 in the proximal direction removes the constraint around the clot treatment component 150 such that it can expand (e.g., automatically expand) from the first state (FIG. 1A) to and/or at least partially toward the second state (FIG. 1B). The clot treatment component 150 may be configured to expand automatically due to material properties such as memory (e.g., shape memory), mechanically in response to application of a tensile or compressive force, via a balloon, and/or another suitable configuration. With the clot treatment component 150 in the second state and/or the catheter 100 in the second configuration 101b, the catheter 100 can be used to treat/remove clot material within a patient.

The catheter 100 can be returned from the second configuration 101b to the first configuration 101a by moving the outer sheath 110 in the distal direction and/or toward the distal terminus 136, for example, by moving the outer sheath drive component 120 in the distal direction. The clot treatment component 150 can be configured to return to the first state (FIG. 1A) as the catheter 100 is transitioned from the second configuration 101b toward the first configuration 101a. In at least some embodiments, for example, the proximal end portion 152b of the clot treatment component 150 can be radially sloped or tapered such that, as the outer sheath 110 is moved distally, the proximal end portion 152b can be received within the outer sheath lumen 114 and continued distal movement of the outer sheath 110 can collapse, enclose, and/or otherwise sheath the clot treatment component 150 (e.g., within the outer sheath 110). Additionally, or alternatively, the clot treatment component 150 can be sheathed by applying a tensile or compressive force to the clot treatment component 150, such as by moving the guidewire sheath 130 relative to the outer sheath 110 when the clot treatment component 150 is coupled to one or both of these sheaths 110, 130.

Additionally, or alternatively, the catheter 100 can be transitioned between the first and second configurations 101a, 101b by moving the guidewire sheath 130 relative to the outer sheath 110. In at least some embodiments, for example, the guidewire sheath drive component 140 can be moved distally relative to the outer sheath 110 to move the guidewire sheath 130 in the distal direction and/or outwardly from within the distal end portion 112a of the outer sheath 110. This can transition the catheter 100 from the first configuration 101a to and/or at least partially toward the second configuration 101b. As the guidewire sheath 130 moves distally, the clot treatment component 150 can be removed from within the outer sheath lumen 114 to expand/deploy, as described above. The catheter 100 can also be transitioned from the second configuration 101b to the first configuration 101a by moving the guidewire sheath drive component 140 in the proximal direction to move the guidewire sheath 130 proximally. This can reposition the clot treatment component 150 within the outer sheath lumen 114, as described above. In these and other embodiments, the catheter 100 can be transitioned between the first and second configurations 101a, 101b by moving the outer sheath drive component 120 and the guidewire sheath drive component 140 in combination, for example, sequentially and/or concurrently.

FIGS. 2A and 2B are enlarged side views of a portion of the catheter 100 of FIGS. 1A and 1B in the first configuration 101a and the second configuration 101b, respectively, in accordance with embodiments of the present technology. In FIGS. 2A and 2B, aspects of the catheter 100 are illustrated as transparent for clarity. The guidewire sheath 130 can include a distal segment 231a, a proximal segment 231b opposite the distal segment 231a, and a third segment 233 (which can also be referred to as an “intermediate segment,” a “medial segment,” a “transition segment,” a “bridging segment,” and the like) extending between the distal segment 231a and the proximal segment 231b. The distal segment 231a can be generally or substantially aligned/coincident with a longitudinal axis L of the catheter 100, such that the distal segment 231a can be generally or substantially concentric with the catheter 100 and/or centered within the outer sheath lumen 114. The proximal segment 231b can be offset with respect to the axis of the distal segment 231a, and the third segment 233 can be an angled intermediate segment that provides a sufficiently smooth transition between the distal segment 231a and the proximal segment 231b for tracking along a guidewire (not shown). The clot treatment component 150 (FIGS. 1A and 1B) can be coupled to the distal segment 231a, such that the clot treatment component 150 can be centered within the outer sheath lumen 114. In some aspects of the present technology, centering the clot treatment component 150 within the outer sheath lumen 114 is expected to improve an operator’s ability to repeatedly and/or consistently unsheathe and re-sheathe the clot treatment component 150 (e.g., out from or back within the outer sheath 110) during a clot treatment procedure, as described in detail above with reference to FIGS. 1A and 1B.

The proximal segment 231b can be generally or substantially parallel to the longitudinal axis L. In at least some embodiments, for example, the proximal segment 231b is not concentric/colinear with the longitudinal axis L and/or the distal segment 231a, as shown in FIGS. 2A and 2B. The outer sheath drive component 120 can be generally or substantially parallel to the proximal segment 231b and/or on an opposite side of the longitudinal axis L from the proximal segment 231b. Although the outer sheath drive component 120 and the guidewire sheath 130 are shown spaced apart in the embodiment illustrated in FIGS. 2A and 2B for illustrative clarity, it will be appreciated that, in practice, the outer sheath drive component 120 may or may not contact the guidewire sheath 130. The outer sheath drive component 120 can be sized such that a region 221 of the outer sheath drive component 120 maintains an overlapping alignment with at least part of the guidewire sheath lumen 134. Accordingly, the outer sheath drive component 120 can be at least partially coextensive, conterminous, and/or otherwise positioned alongside with the guidewire sheath lumen 134 when the catheter 100 is in the first and second configurations 101a, 101b. In some embodiments, for example, the region 221 can be coextensive and/or conterminous with at least part of the proximal segment 231b when the catheter 100 is in the first and second configurations 101a, 101b (FIGS. 1A and 1B, respectively). In some aspects of the present technology, the coextensive and/or conterminous alignment between the outer sheath drive component 120 and the guidewire sheath 130 is expected to improve the user’s ability to repeatedly and/or consistently unsheathe and re-sheathe the clot treatment component 150 during a clot treatment procedure while also maintaining an open lumen through the catheter 100 (e.g., the guidewire sheath lumen 134). For example, the coextensive and/or conterminous relationship between the proximal segment 231b of the guidewire sheath 130 and the region 221 of the outer sheath drive component 120 in the second configuration 101b can maintain the guidewire sheath 130 and the outer sheath drive component 120 in a slidable relationship relative to each other and allow the guidewire sheath 130 to be moved proximally to return the catheter 100 to the first configuration 101a. The relatively short length of the guidewire sheath 130 is expected to allow the user to reload the guidewire and/or insert other devices along the guidewire during the clot treatment procedure. In other embodiments, the region 221 of the outer sheath drive component 120 may not be in overlapping alignment with at least part of the guidewire sheath lumen 134 when the catheter 100 is in the first and second configurations 101a, 101b, but can still maintain advantages described above.

The intermediate segment 233 can be angled or sloped relative to the longitudinal axis L as described above. For example, the intermediate segment 233 can be angled radially inward, for example, toward the longitudinal axis L from the proximal segment 231b toward the distal segment 231a and/or toward the guidewire sheath drive component 140. The intermediate segment 233 can connect the distal and proximal segments 231a, 231b, such that the guidewire sheath lumen 134 and/or a guidewire (not shown) can extend fully through the guidewire sheath 130 as described above. Additionally, or alternatively, the guidewire sheath drive component 140 can be operably coupled to the guidewire sheath 130 at or near the intermediate segment 233.

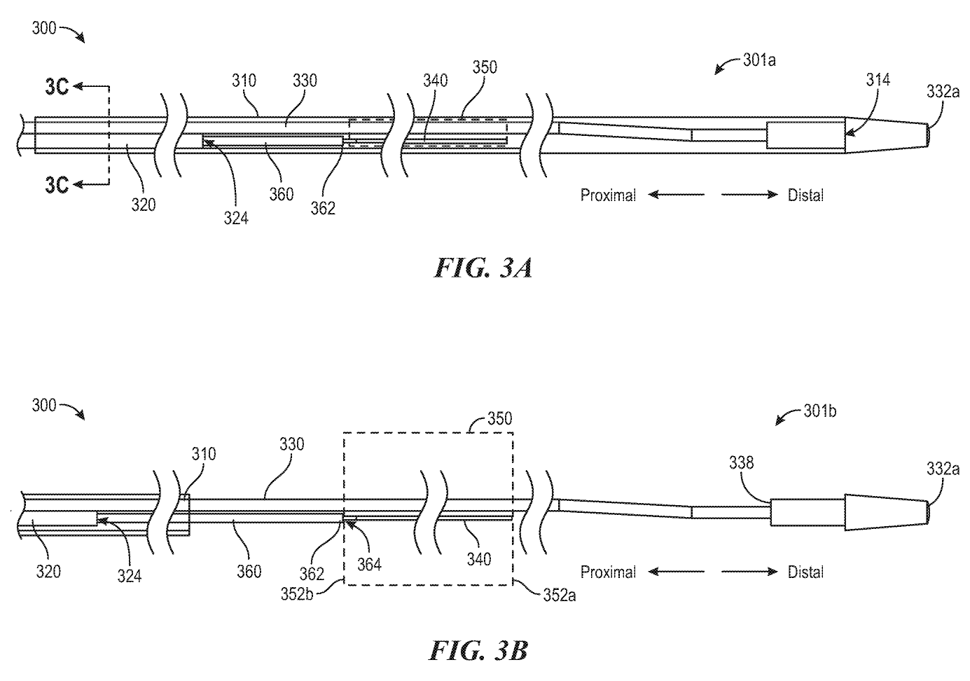

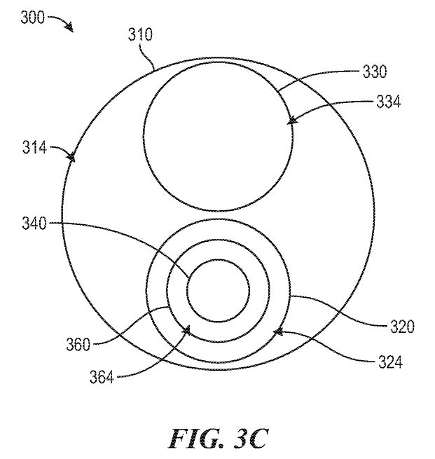

FIGS. 3A and 3Bare side views of another clot treatment and/or clot removal catheter 300 (“catheter 300”) in a first configuration 301a and a second configuration 301b, respectively, in accordance with embodiments of the present technology. In FIGS. 3A and 3B, aspects of the catheter 300 are illustrated as transparent for clarity. FIG. 3C is a partially-schematic cross-sectional view of the catheter 300 taken along line 3C–3C of FIG. 3A. The catheter 300 can include some aspects that are generally similar or identical in structure and/or function to aspects of the catheter 100 of FIGS. 1A-2B, with like reference numbers (e.g., outer sheath 310 versus the outer sheath 110 of FIGS. 1A-2B) indicating generally similar or identical aspects. Additionally, in the illustrated embodiment, the catheter 300 includes a clot treatment drive component 360. The clot treatment drive component 360 can be positioned (e.g., radially, telescopically, and the like) between the outer sheath drive component 320 and the guidewire sheath drive component 340, such that each of the outer sheath drive component 320, the guidewire sheath drive component 340, and the clot treatment drive component 360 can be moved independently in the proximal direction and/or distal direction. In the illustrated embodiment, for example, the guidewire sheath drive component 340 is slidably disposed within the clot treatment drive component lumen 364 (FIG. 3B) and the clot treatment drive component 360 is slidably disposed within the outer sheath drive component lumen 324.

The clot treatment drive component 360 can include an elongated tube or member having a distal end portion 362 and a lumen 364 (“clot treatment drive component lumen 364”). The clot treatment drive component 360 can be operably coupled to a clot treatment component 350 (shown schematically in FIGS. 3A and 3B). In at least some embodiments, for example, the distal end portion 362 of the clot treatment drive component 360 can be coupled to a proximal end portion 352b of the clot treatment component 350, such as shown in FIG. 3B. In these and other embodiments, any other suitable portion or end of the clot treatment drive component 360 can be coupled to any other suitable portion or end of the clot treatment component 350. Additionally, or alternatively, any other suitable portion or end of the clot treatment component 350 (e.g., distal end portion 352a) can be coupled to the guidewire sheath 330 and/or a proximal end portion 338 of a distal tip 332a of the guidewire sheath 330.

During a clot treatment procedure, the outer sheath drive component 320 and/or the guidewire sheath drive component 340 can be moved to transition the catheter 300 between the first configuration 301a and the second configuration 301b, such as described in detail above with reference to FIGS. 1A-2B. Additionally, the clot treatment drive component 360 and/or guidewire sheath drive component 340 can be moved to transition the clot treatment component 350 between the first state (FIG. 3A) and the second state (FIG. 3B). In at least some embodiments, for example, the clot treatment drive component 360 can be moved distally to expand/deploy the clot treatment component 350 from the first (e.g., delivery) state to and/or at least partially toward the second (e.g., deployed) state. In other embodiments, the clot treatment drive component 360 can be moved proximally to expand/deploy the clot treatment component 350 from the first state to and/or at least partially toward the second state. Additionally, or alternatively, the clot treatment drive component 360 can be moved distally to return the clot treatment component 350 the second state to and/or at least partially toward the first state, for example, to position/sheathe the clot treatment component 350 within the outer sheath lumen 314, as shown in FIG. 3A. In other embodiments, the clot treatment drive component 360 can be moved proximally to return the clot treatment component 350 the second state to and/or at least partially toward the first state. In these and other embodiments, the clot treatment component 350 can automatically transition between the first and second states, such as described in detail above regarding FIGS. 1A and 1B. In at least some embodiments, for example, the clot treatment component 350 can be biased toward one state (e.g., the second state), and the clot treatment drive component 360 and/or guidewire sheath drive component 340 can be moved to control the degree and/or rate that the clot treatment component 350 transitions toward the biased state. In these and other embodiments, the clot treatment drive component 360 and/or guidewire sheath drive component 340 can be moved to adjust (e.g., increase and/or decrease) one or more dimensions (e.g., length, width, diameter, cross-sectional area, and the like) and/or adjust one or more characteristics/mechanical properties (e.g., radial force, tension, compression, shape, and the like) of the clot treatment component 350. For example, moving the clot treatment drive component 360 distally to expand/deploy the clot treatment component 350, as described above, may also cause a length of the clot treatment component 350 to decrease and/or a cross-sectional area of the clot treatment component 350 to increase.

Although the catheter 300 includes one clot treatment drive component 360 in the embodiment illustrated in FIGS. 3A-3C, in other embodiments the catheter 300 can include more clot treatment drive components, such as at least two, three, four, five, or any other suitable number of clot treatment drive components. Each additional clot treatment drive component can be positioned between (e.g., radially between, telescopically, and the like) the outer sheath drive component 320 and the guidewire sheath drive component 340, at least generally similar to the position of the clot treatment drive component 360. For example, a second clot treatment drive component (not shown in FIGS. 3A-3C) can be slidably disposed within the clot treatment drive component lumen 364, between an interior surface of the clot treatment drive component 360 and an exterior surface of the guidewire sheath drive component 340. Each additional clot treatment drive component can be operably coupled to a respective portion or end of the clot treatment component 350, such that each additional clot treatment drive component can increase the user’s ability to adjust/control the clot treatment component 350. In at least some embodiments, for example, each additional clot treatment drive component can be configured to adjust at least one dimension of the clot treatment component 350.

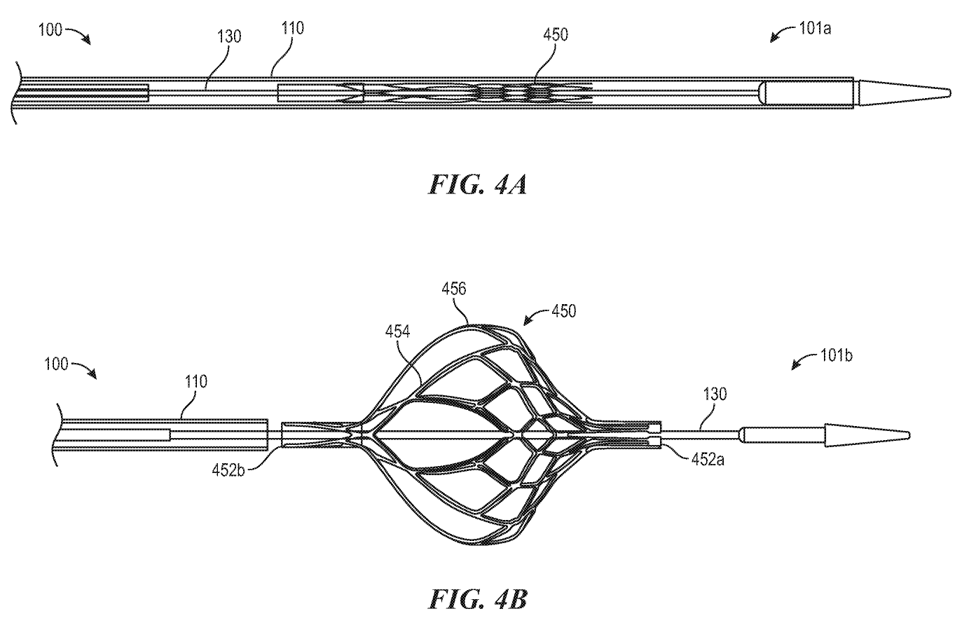

FIGS. 4A and 4B are side views of the clot treatment catheter 100 in the first configuration 101a and the second configuration 101b, respectively, in accordance with embodiments of the present technology. In FIGS. 4A and 4B, aspects of the catheter 100 are illustrated as transparent for clarity. In the illustratedembodiment, the catheter 100 includes a clot treatment component 450 having a plurality of struts 454. Individual ones of the struts 454 can extend at least partially between the distal end portion 452a and the proximal end portion 452b of the clot treatment component 450. In the illustrated embodiment, the proximal end portion 452b is coupled to (e.g., configured to move with) the guidewire sheath 130 and the distal end portion 452a is configured to move/slide along the guidewire sheath 130. When the catheter 100 is in the second configuration 101b, the clot treatment component 450 can be expanded/deployed radially outward from the guidewire sheath 130. In some embodiments, for example, the struts 454 can be shape-memory struts and can automatically expand/deploy in response to movement of the guidewire sheath 130 and/or the outer sheath 110. The clot treatment component 450 can further include an apex or radially-extended portion 456 that defines a maximum outer dimension (e.g., width, radius, etc.) of the clot treatment component 405. When expanded/deployed, the clot treatment component 450 can be used to remove clot material from a within patient. Additional details regarding the clot treatment component can be found in U.S. Pat. Nos. 8,784,434, the entirety of which was previously incorporated by reference herein. Although the catheter 100 is carrying a single clot treatment component 450 in the embodiment shown in FIGS. 4A and 4B, in other embodiments, the catheter 100 can carrying multiple clot treatment components.

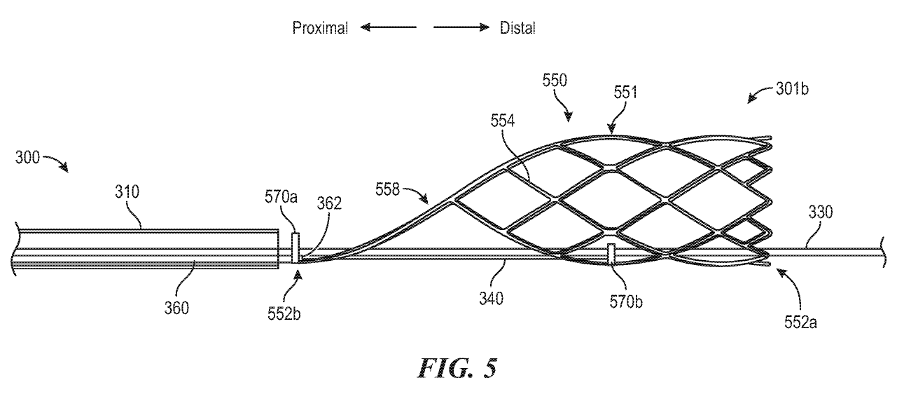

FIG. 5 is a side view of the clot treatment catheter 300 in the second configuration 301b in accordance with embodiments of the present technology. In FIG. 5, aspects of the catheter 300 are illustrated as transparent for clarity. In the illustrated embodiment, the catheter 300 includes a clot treatment component 550 including a coring element 551. The coring element 551 includes a cylindrical fenestrated structure defined by a plurality of struts 554. Individual ones of the struts 554 can extend at least partially between a distal end portion 552a and a proximal end portion 552b of the coring element 551. The coring element 551 can include a leading edge 558 at or near the proximal end portion 552b and configured to core or otherwise remove clot material from within a patient. For example, the coring element 551 can be expanded distal from clot material and retracted proximally such that the leading edge 558 engages the clot material to thereby capture and/or otherwise remove at least a portion of the clot material. In some embodiments, the clot treatment component 550 further includes an expandable bag or cylindrical portion coupled to the distal portion 552a of the coring element 551 and extending distally therefrom. The expandable bag can be a braided and/or mesh structure (e.g., a braided filament mesh structure) and can be configured to capture and retain the clot material after it is cored by the coring element 551. In some embodiments, a distal portion of the expandable bag can be coupled to the distal tip 332a (FIGS. 3A and 3B) of the guidewire sheath 330 such that movement of the guidewire sheath 330 relative to the coring element 551 can lengthen/shorten the expandable bag. Additional details regarding the clot treatment component 550 can be found in U.S. Pat. No. 10,342,571 and/or U.S. Pat. No. 10,912,577, the entireties of which were both previously incorporated by reference herein.

In the illustrated embodiment, the proximal end portion 552b of the coring element 551 is coupled to the clot treatment drive component 360. Specifically, the catheter 300 includes a first coupling component 570a configured to couple the proximal end portion 552b of the coring element 551 to the distal end portion 362 of the clot treatment drive component 360. With continued reference to the illustrated embodiment, the catheter 300 can include a second coupling component 570b configured to couple the coring element 551 to the guidewire sheath drive component 340 and/or the guidewire sheath 330. In other embodiments, the catheter 300 can include another clot treatment drive component (not shown in FIG. 5) coupled to the coring element 551 via the second coupling component 570b. In embodiments where the first and second coupling components 570a-b are both coupled to the coring element 551, the components/sheaths associated with the first and second coupling components 570a-b can be moved relative to each other to manipulate the coring element 551. For example, the distance between the first and second coupling components 570a-b can be decreased (e.g., by distally advancing the clot treatment drive component 360 and/or proximally retracting the guidewire sheath drive component 340 and/or the guidewire sheath 330) to cause the coring element 551 to shorten longitudinally and/or expand radially outward from the guidewire sheath 330. As another example, the distance between the first and second coupling components 570a-b can be increased (e.g., by proximally retracting the clot treatment drive component 360 and/or distally advancing the guidewire sheath drive component 340 and/or the guidewire sheath 330) to cause the coring element 551 to lengthen longitudinally and/or compress radially inwardly toward the guidewire sheath 330. In at least some embodiments, at least part of the clot treatment component 550 (e.g., the expandable bag described above) can be coupled to the distal tip 332a (FIGS. 3A and 3B) of the guidewire sheath 330 such that (i) moving the guidewire sheath 330 distally can lengthen the clot treatment component 550 longitudinally and/or compress the clot treatment component 550 radially inwardly toward the guidewire sheath 330 and (ii) moving the guidewire sheath 330 proximally can shorten the clot treatment component 550 longitudinally and/or expand the clot treatment component 550 radially outward away from the guidewire sheath 330, in addition to or in lieu of movement of one or more of the other sheaths/components of the catheter 300.

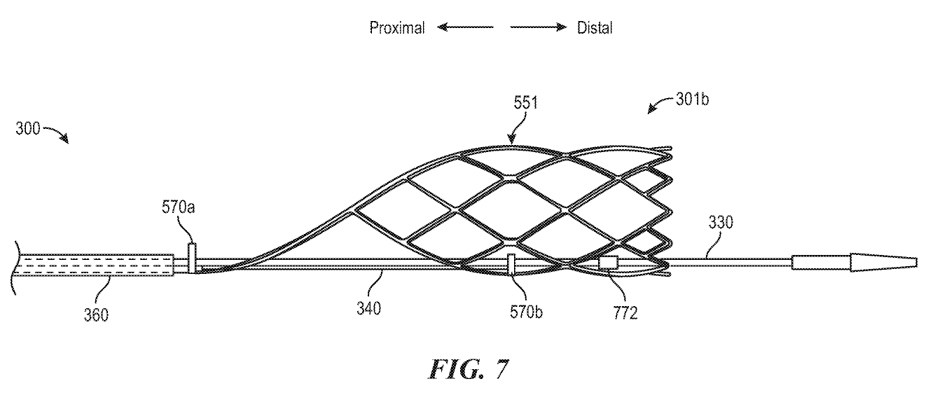

FIG. 7 is a side view of the clot treatment catheter 300 in the second configuration 301b in accordance with embodiments of the present technology. In FIG. 7, aspects of the catheter 300 are illustrated as transparent for clarity. In the illustrated embodiment, the catheter 300 includes the coring element 551 described previously with reference to FIG. 5. In the embodiment illustrated in FIG. 7, however, the second coupling component 570b is not coupled to the guidewire sheath drive component 340. Instead, the guidewire sheath drive component 340 is coupled to a drive cuff 772 (e.g., a stop member) positioned around the guidewire sheath 330 distally of the second coupling component 570b. The guidewire sheath drive component 340 can be slidably disposed through the second coupling component 570b such that the drive cuff 772 and/or the second coupling component 570b can be moved relative to one another. This relative movement of the drive cuff 772 and the second coupling component 570b can be used to change a configuration of the coring element 551 and/or the clot treatment component 550. For example, the drive cuff 772 can be moved proximally (e.g., by proximally retracting the guidewire sheath drive component 340 and/or the guidewire sheath 330) to contact the second coupling component 570b and cause the second coupling component 570b to move proximally. This, in turn, can decrease the distance between the first coupling component 570a and the second coupling component 570b and thereby cause the coring element 551 to shorten longitudinally and/or expand radially outward from the guidewire sheath 330. In some embodiments, the guidewire sheath drive component 340 can be biased in the proximal direction, such that the drive cuff 772 is biased proximally into engagement with the second coupling component 570b to cause the coring element 551 to shorten and/or expand radially outwardly from the guidewire sheath 330.

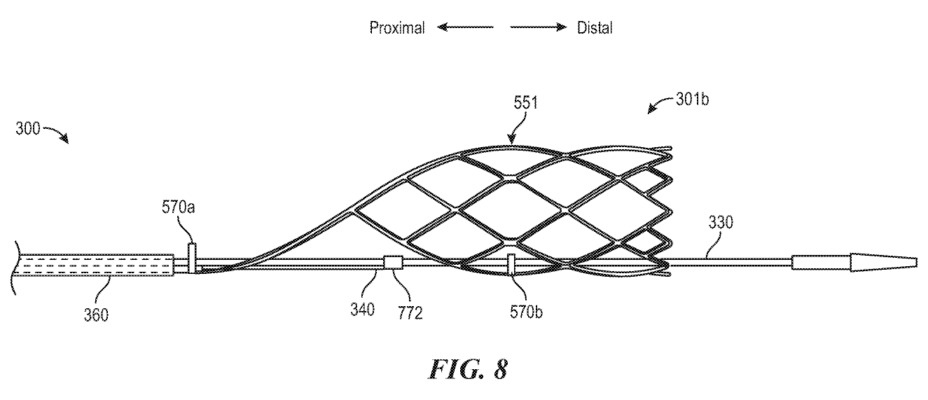

FIG. 8 is a side view of the clot treatment catheter 300 in the second configuration 301b in accordance with embodiments of the present technology. In FIG. 8, aspects of the catheter 300 are illustrated as transparent for clarity. The catheter 300 illustrated in FIG. 8 can include at least some features that are at least generally similar or identical in structure and/or function to the catheter 300 described previously with reference to FIG. 7. However, in the embodiment illustrated in FIG. 8, the drive cuff 772 is positioned around the guidewire sheath 330 proximally of the second coupling component 570b. Accordingly, the drive cuff 772 can be moved distally (e.g., by distally advancing the guidewire sheath drive component 340 and/or the clot treatment drive component 360) to contact the second coupling component 570b and increase the distance between the first coupling component 570a and the second coupling component 570b to cause the coring element 551 to lengthen longitudinally and/or compress radially inwardly toward the guidewire sheath 330. In some embodiments, the guidewire sheath drive component 340 can be biased in the distal direction, such that the drive cuff 772 is biased distally into engagement with the second coupling component 570b to cause the coring element 551 to lengthen and/or contract radially inwardly toward the guidewire sheath 330.

Accordingly, in some aspects of the present technology, a catheter can include an outer sheath, an outer sheath drive component operably coupled to the outer sheath, a guidewire sheath slidably disposed within the outer sheath, and a guidewire sheath drive component operably coupled to the guidewire sheath and slidably disposed within the outer sheath drive component. The outer sheath drive component can be moved proximally and/or distally to cause corresponding movement of the outer sheath. The guidewire sheath drive component can be moved proximally and/or distally to cause corresponding proximal and/or distal movement of the guidewire sheath. The movement of the outer sheath and/or the guidewire sheath can transition the catheter between a first configuration and a second configuration. In some embodiments, the catheter includes a clot treatment component carried within the outer sheath, and, during a procedure, the clot treatment component can be deployed from the outer sheath and/or repositioned within the outer sheath by moving one or both of the outer sheath drive component and the guide wire sheath drive component. For example, the clot treatment component can be carried within the outer sheath when the catheter is in the first configuration, and the clot treatment component can be deployed from the outer sheath when the catheter is in the second configuration. In these and other embodiments, the catheter can include at least one clot treatment drive component operably coupled to the clot treatment component. The clot treatment drive component can be positioned between (e.g., radially between, telescopically between, and the like) the outer sheath drive component and the guidewire sheath drive component. During a procedure, the clot treatment drive component can be moved proximally and/or distally to deploy, sheath, and/or change at least one dimension (e.g., length, width, diameter, cross-sectional area, and the like) of the clot treatment component.

Several aspects of the present technology are set forth in the following examples:

1. An intravascular catheter for treatment of clot material from within a blood vessel of a human patient, comprising: an outer sheath having an outer sheath lumen; an outer sheath drive component having an outer sheath drive component lumen, wherein the outer sheath drive component is operably coupled to the outer sheath; a guidewire sheath including a guidewire sheath lumen configured to receive a guidewire, wherein at least a portion of the guidewire sheath is slidably disposed within the outer sheath lumen; a guidewire sheath drive component slidably disposed within the outer sheath drive component lumen, wherein the guidewire sheath drive component is operably coupled to the guidewire sheath; and a clot treatment component coupled to the guidewire sheath, wherein the clot treatment component is configured to transition between (i) a first configuration in which the clot treatment component is positioned within the outer sheath lumen, and (ii) a second configuration in which at least a portion of the clot treatment component is exposed from the outer sheath lumen in response to movement of the outer sheath drive component, the guidewire sheath drive component, or both the outer sheath drive component and the guidewire sheath drive component.

2. The catheter of example 1, further comprising a clot treatment drive component slidably disposed within the outer sheath drive component lumen and operably coupled to the clot treatment component, wherein the clot treatment component is configured to transition between the first configuration and the second configuration in response to movement of the clot treatment drive component, the outer sheath drive component, the guidewire sheath drive component, or any two or more of the outer sheath drive component, the guidewire sheath drive component, and clot treatment drive component.

3. The catheter of example 1 or example 2 wherein the outer sheath drive component includes a region configured to overlap with a portion of the guidewire sheath when the clot treatment component is in the first configuration and the second configuration.

4. The catheter of example 3 wherein the guidewire sheath includes a proximal segment and a distal segment, wherein at least part of the proximal segment is configured to be conterminous with the portion of the outer sheath drive component, and wherein the distal segment is centered within the outer sheath lumen.

5. The catheter of example 4 wherein at least a portion of the clot treatment component is coupled to the distal segment of the guidewire sheath.

6. The catheter of example 5 wherein the distal segment of the guidewire sheath includes a distal tip of the catheter, and wherein at least the portion of the clot treatment component is coupled to the distal tip.

7. The catheter of any of examples 4–6 wherein at least a portion the clot treatment component is slidably disposed on the distal segment.

8. The catheter of any of examples 4–7 wherein the guidewire sheath further includes an intermediate segment extending between the distal segment and the proximal segment, wherein the intermediate segment is sloped radially inward from the proximal segment toward the distal segment.

9. The catheter of example 1 wherein the guidewire sheath includes a distal segment centered within the outer sheath lumen, and wherein at least a portion of the clot treatment component is coupled to the distal segment of the guidewire sheath.

10. The catheter of example 9 wherein the distal segment of the guidewire sheath includes a distal tip of the catheter, and wherein at least the portion of the clot treatment component is coupled to the distal tip.

11. The catheter of example 9 or example 10 wherein at least a portion the clot treatment component is slidably disposed on the distal segment of the guidewire sheath.

12. The catheter of any of examples 9–11 wherein the guidewire sheath further includes a proximal segment positioned radially outwardly from the distal segment of the guidewire sheath.

13. The catheter of example 12 wherein the proximal segment is not centered within the outer sheath lumen.

14. The catheter of example 12 or example 13 wherein the guidewire sheath further includes an intermediate segment extending between the distal segment and the proximal segment, wherein the intermediate segment is sloped radially inward from the proximal segment toward the distal segment.

15. A method for intravascular treatment of clot material from within a blood vessel of a human patient, the method comprising: advancing a catheter distally through the blood vessel; and transitioning the catheter from (i) a first configuration in which a clot treatment component of the catheter is positioned within an outer sheath lumen of an outer sheath of the catheter, and (ii) a second configuration in which at least a portion of the clot treatment component is exposed from the outer sheath lumen, wherein the catheter includes (a) an outer sheath drive component including an outer sheath drive component lumen and coupled to the outer sheath and (b) an elongate member operably coupled to the clot treatment component and slidably disposed within the outer sheath drive component lumen, and wherein transitioning the catheter includes at least one of— moving the outer sheath drive component in a first direction relative to the clot treatment component to cause the outer sheath to uncover at least the portion of the clot treatment component, and/or moving the elongate member in a second direction relative to the outer sheath to cause at least the portion of the clot treatment component to extend beyond the outer sheath lumen.

16. The method of example 15 wherein the second direction is opposite the first direction.

17. The method of example 15, further comprising causing the clot treatment component to expand when the catheter is in the second configuration.

18. The method of example 17 wherein causing the clot treatment component to expand includes transitioning the catheter to the second configuration to allow the clot treatment component to expand.

19. The method of example 17 wherein causing the clot treatment component to expand includes moving a clot treatment drive component operably coupled to the clot treatment component relative to a guidewire sheath positioned within the catheter and adjacent to the outer sheath drive component.

20. The method of example 17 wherein causing the clot treatment component to expand includes moving a drive cuff of the catheter to cause the expansion of the clot treatment component.

21. The method of example 20 wherein moving the drive cuff includes moving the drive cuff in the first direction relative to the clot treatment component to drive the expansion of the clot treatment component.

22. The method of example 20 wherein moving the drive cuff includes moving the drive cuff in the first direction relative to the clot treatment component to allow the clot treatment component to expand.

23. The method of any of examples 15-22, wherein the elongate member includes a guidewire sheath drive component, and wherein moving the elongate member includes moving the guidewire sheath drive component in the second direction relative to the outer sheath to cause at least the portion of the clot treatment component to extend beyond the outer sheath lumen.

24. The method of example 23 wherein moving the guidewire sheath drive component includes causing movement of a guidewire sheath operably coupled to the guidewire sheath drive component, and wherein the clot treatment component is operably coupled to the guidewire sheath so that causing movement of the guidewire sheath includes causing at least the portion of the clot treatment component to extend beyond the outer sheath lumen.

25. A catheter for treating a human patient, the catheter comprising: an outer sheath having an outer sheath lumen; an outer sheath drive component having an outer sheath drive component lumen, wherein the outer sheath drive component is slidably disposed within the outer sheath lumen and operably coupled to the outer sheath; a guidewire sheath including a guidewire sheath lumen configured to receive a guidewire, wherein at least a portion of the guidewire sheath is slidably disposed within the outer sheath and laterally adjacent to the outer sheath drive component; and a guidewire sheath drive component slidably disposed within the outer sheath drive component, wherein the guidewire sheath drive component is operably coupled to the guidewire sheath; a clot treatment component coupled to the guidewire sheath; and a clot treatment drive component slidably disposed within the outer sheath drive component lumen and operably coupled to the clot treatment component, wherein— the catheter is configured to transition between a (i) first configuration in which the clot treatment component is positioned within the outer sheath lumen, and (ii) a second configuration in which at least a portion of the clot treatment component is exposed from the outer sheath lumen in response to movement in response to at least one of (a) first movement the outer sheath drive component relative to the guidewire sheath in a first direction, and (b) second movement of the guidewire sheath drive component relative to the outer sheath in a second direction, and the clot treatment drive component is configured to transition the clot treatment component between (i) a first state in which the clot treatment component has a first dimension, and (ii) a second state in which the clot treatment component has a second dimension greater or less than the first dimension.

26. The catheter of example 25 wherein: in the first state, the clot treatment component has a first mechanical property, and in the second state, the clot treatment component has a second mechanical property.

27. The catheter of example 26 wherein: the first mechanical property includes at least one of a first radial force, a first tensile force, a first compressive force, and/or a first shape, and the second mechanical property includes at least one of a second radial force, a second tensile force, a second compressive force, and/or a second shape.

28. The catheter of any of examples 25–27 wherein the clot treatment component includes a mechanical thrombectomy component extending radially outwardly from the guidewire sheath.

29. The catheter of any of examples 25–27 wherein the clot treatment component includes a coring element having a proximally-facing leading edge.

30. The catheter of any of examples 25–29 wherein the clot treatment drive component is a first clot treatment drive component operably coupled to a first portion of the clot treatment component, the catheter further comprising a second clot treatment drive component slidably coupled to a second portion of the clot treatment component.

31. The catheter of example 30 wherein the first clot treatment drive component includes a first clot treatment drive component lumen, and wherein the second clot treatment drive component is slidably disposed within the first clot treatment drive component lumen.

32. The catheter of any of examples 25–31 wherein the second direction is opposite the first direction.

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We Claim:

1. A catheter for treating clot material within a blood vessel of a patient, comprising:
an outer sheath;
an outer sheath drive member coupled to the outer sheath;
a guidewire sheath configured to receive a guidewire;
a guidewire sheath drive member positioned within the outer sheath drive member and coupled to the guidewire sheath; and
a medical device coupled to the guidewire sheath, wherein the medical device has a compressed position within the outer sheath and an expanded position at least partially outside the outer sheath, and wherein (a) the outer sheath drive member is movable to proximally retract the outer sheath relative to the guidewire sheath to move the medical device from the compressed position to the expanded position and/or (b) the guidewire sheath drive member is movable to distally advance the guidewire sheath relative to the outer sheath to move the medical device from the compressed position to the expanded position.

2. The catheter of claim 1 wherein a distal portion of the outer sheath drive member is fixedly coupled to the outer sheath.

3. The catheter of claim 1 wherein a distal portion of the guidewire sheath drive member is fixedly coupled to the guidewire sheath.

4. The catheter of claim 1 wherein the guidewire sheath has a distal portion fixedly coupled to an atraumatic tip and a proximal portion opposite the distal portion, wherein the outer sheath has a distal portion and a proximal portion opposite the distal portion, and wherein the atraumatic tip abuts the distal portion of the outer sheath when the medical device is in the compressed position.

5. The catheter of claim 1 wherein the guidewire sheath has a distal portion and a proximal portion opposite the distal portion, wherein the outer sheath has a distal portion and a proximal portion opposite the distal portion, and wherein the proximal portion of the guidewire sheath is positioned within the outer sheath distal to the proximal portion of the outer sheath when the medical device is in the expanded position.

6. The catheter of claim 1 wherein—
the outer sheath extends along a longitudinal axis,
the guidewire sheath has a proximal portion, a distal portion, and an intermediate portion between the proximal portion and the distal portion,
the distal portion is concentric with the longitudinal axis, and
the proximal portion is radially offset from the longitudinal axis.

7. The catheter of claim 6 wherein the intermediate portion is angled radially inward toward the longitudinal axis from the proximal portion to the distal portion.

8. The catheter of claim 6 wherein the medical device is coupled to the distal portion of the guidewire sheath.

9. The catheter of claim 1, further comprising a medical device drive member positioned within the outer sheath drive member and operably coupled to the medical device, wherein the guidewire sheath drive member is positioned within the medical device drive member.

10. The catheter of claim 9 wherein a distal portion of the medical device drive member is fixedly coupled to a proximal portion of the medical device.

11. The catheter of claim 10 wherein the medical device drive member and the guidewire sheath drive member are movable relative to one another with the medical device in the expanded position to further expand the medical device.

12. The catheter of claim 1 wherein a proximal portion of the medical device is fixedly coupled to the guidewire sheath.

13. The catheter of claim 1 wherein the medical device comprises a thrombectomy device comprising a unitary structure formed by a plurality of interconnected struts.

14. A catheter for treating clot material within a blood vessel of a patient, comprising:
a first sheath;
a first drive member operably coupled to the first sheath;
a second sheath positioned at least partially within the first sheath and configured to receive a guidewire;
a second drive member extending through the first drive member and operably coupled to the second sheath; and
a medical device coupled to the second sheath, wherein the first drive member and the second drive member are movable relative to one another to deploy the medical device from (a) a compressed position within the first sheath to (b) an expanded position at least partially outside the first sheath.

15. The catheter of claim 14 wherein a distal portion of the first drive member is fixedly coupled to the first sheath, and wherein a distal portion of the second drive member is fixedly coupled to the second sheath.

16. The catheter of claim 15 wherein a proximal portion of the first drive member is operably coupled to a handle, wherein a proximal portion of the second drive member is operably coupled to the handle, and wherein the handle is configured to move the first drive member relative to the second drive member and/or to move the second drive member relative to the first drive member.

17. The catheter of claim 16 wherein the first sheath has a proximal end portion positioned distal to the handle, and wherein the second sheath has a proximal end portion positioned distal to the handle.

18. A method of treating clot material within a blood vessel of a patient, the method comprising:
advancing a catheter assembly distally through the blood vessel in a first configuration along a guidewire to proximate the clot material, wherein the guidewire extends through a guidewire sheath of the catheter assembly, wherein the guidewire sheath is coupled to a clot treatment device, and wherein in the first configuration the clot treatment device is positioned within an outer sheath of the catheter assembly between the outer sheath and the guidewire sheath;
deploying the clot treatment device from the outer sheath to a second configuration by (a) moving an outer sheath drive member coupled to the outer sheath relative to a guidewire sheath drive member coupled to the guidewire sheath and/or (b) moving the guidewire sheath drive member relative to the outer sheath drive member, wherein the guidewire sheath drive member is positioned within the outer sheath drive member; and
engaging the clot treatment device with the clot material within the blood vessel.

19. The method of claim 18 wherein engaging the clot treatment device with the clot material comprises proximally retracting the clot treatment device through the clot material to at least partially capture the clot material.

20. The method of claim 18 wherein deploying the clot treatment device from the outer sheath to the second configuration comprises (a) proximally retracting the outer sheath drive member relative to the guidewire sheath drive member and/or (b) distally advancing the guidewire sheath drive member relative to the outer sheath drive member.
